(19) United States
(12) Patent Application Publication
(10) Pub. No.: US 2025/0352742 A1
(43) Pub. Date: Nov. 20, 2025

(54) METERED DOSE INHALER CANISTER WITH IMPROVED SEALING ARRANGEMENT

(71) Applicants: John P. BUNTING, Woodbury, MN (US); Lee HODGES, Woodbury, MN (US); Stephen HOWGILL, Woodbury, MN (US); Kindeva Drug Delivery L.P., Woodbury, MN (US)

(72) Inventors: John P. Bunting, Castle Donington (GB); Lee Hodges, Clitheroe (GB); Stephen Howgill, Leicester (GB)

(21) Appl. No.: 18/867,731

(22) PCT Filed: May 24, 2023
(86) PCT No.: PCT/US2023/023390

§ 371 Date: Nov. 20, 2024

(30) Foreign Application Priority Data
May 24, 2022 (GB) 2207610.3

(51) Int. Cl. A61M 15/00; A61M 39/00; A61M 39/22

(52) U.S. Cl. CPC A61M 15/0001; A61M 15/0028; A61M 39/22; A61M 2039/0009; A61M 2205/0216

(57) ABSTRACT

A metered dose inhaler valve (26) has a reservoir portion (37) for assembly with a canister (14) for receiving a pressurised formulation of medicament and propellant, a metering valve body (96) defining a metering chamber, and a metering valve stem (24) having an outlet (56) and being axially moveable within the body. The stem moves between a first position in which the chamber is in fluid communication with the canister, and a second position in which the chamber is in fluid communication with the valve stem outlet. A metering valve seal is defined by the valve body and includes an opening through which the stem passes to form a dynamic seal between the stem and at least one of the outside atmosphere and the pressurized canister, where the opening is adapted to be stretched wider by the stem passing through it than it would be absent the valve stem.

This application claims the benefit of priority under 35 U.S.C. § 119 (a) of GB Application No. 2207610.3, filed May 24, 2022, the disclosure of which is incorporated by reference herein in its entirety.

### TECHNICAL FIELD

The present disclosure concerns a metered dose inhaler (MDI) canister with an improved sealing arrangement. More specifically, the present disclosure concerns a pressurised MDI canister having a sealing arrangement enabling the use of high hardness materials.

### BACKGROUND ART

Delivery of aerosolized medicament to the respiratory tract for the treatment of respiratory and other diseases can be done using, by way of example, pressurized metered dose inhalers (pMDI), dry powder inhalers (DPI), or nebulizers. pMDIs are familiar to many patients who suffer from asthma or from chronic obstructive pulmonary disease (COPD).

pMDI devices can include an aluminium canister, sealed with a metering valve, that contains medicament formulation. Generally, the medicament formulation is a solution and/or suspension of one or more medicinal compounds in a liquefied hydrofluoroalkane (HFA) propellant.

In pulmonary pMDIs, the sealed canister can be provided to the patient in an actuator, which is a generally L-shaped plastic part that includes a generally vertical tube that surrounds the canister plus a generally horizontal tube that forms a patient portion (e.g., a mouthpiece or nosepiece) that can define an inspiration (or inhalation) orifice.

The canister typically includes a metering valve that is crimped onto an appropriately sized metal can. The metal can is typically made of aluminium, having a wall thickness of approximately 0.5 mm. The canister contains a formulation typically including liquid propellant(s), drug(s), co-solvent(s) and excipient(s). To prevent loss of the formulation, (primarily the liquid propellant), the metering valve contains rubber components that form seals.

Historically, the propellants in most pMDIs have been chlorofluorocarbons (CFCs). However, environmental concerns during the 1990s led to the replacement of CFCs with hydrofluoroalkanes (HFAs) as the most used propellant in pMDIs. Although HFAs do not cause ozone depletion they do have a stated high global warming potential (GWP), which is a measurement of the future radiative effect of an emission of a substance relative to that of the same amount of carbon dioxide (CO₂). Various other propellants have been proposed over the years. Among them, carbon dioxide (CO₂) has been mentioned as a potential propellant for pMDIs.

Applicant's application WO 2021/101997 discloses an MDI in which a metering valve stem seal has a Shore D hardness of 45 to 80. Its opening is adapted to be stretched wider by the metering valve stem passing through it than it would be absent the valve stem. This arrangement is suited to MDIs which utilise carbon dioxide (CO₂) as the propellant. It will be noted that WO'997 is a springless design, in which increased pressure of the CO₂ is used to return the stem.

### SUMMARY OF INVENTION

It is an aim of the present disclosure to provide an improved sealing arrangement for the metering valve such that materials with a high hardness of about Shore D 80 can be utilised. The present disclosure seeks, via the use of such materials, to reduce the leachable/extractables profile of pMDIs in addition to providing a valve that has improved compatibility with propellant types such as HFA 134a, 152a, 1234ze, and CO₂. In particular, the present disclosure identifies that it would be beneficial to provide a pMDI having a high-hardness seal in combination with a broader range of propellants, e.g., HFA propellants, in addition to CO₂.

According to a first aspect of the present disclosure there is provided a metered dose inhaler valve, including:
a reservoir portion for assembly with a canister for receiving a pressurised formulation of medicament and propellant;
a metering valve body defining a metering chamber;
a metering valve stem having an outlet and being axially moveable within the metering valve body between a first position in which the metering chamber is in fluid communication with the pressurized canister, and a second position in which the metering chamber is in fluid communication with the valve stem outlet; and
a resilient return member urging the metering valve stem from the second position to the first position;
where:
a metering valve seal is defined by the valve body, the metering valve seal including an opening through which the metering valve stem passes to form a dynamic seal between the metering valve stem and the outside atmosphere; and
the metering valve stem seal opening is adapted to be stretched wider by the metering valve stem passing through it than it would be absent the metering valve stem.

Such a valve is suitable for non-CO₂ propellants provided at lower pressures (5-6 bar), and further provides a valve with beneficial leak performance.

In one or more embodiments, the metering valve stem has a first end and a second end; the outlet is defined at the first end; the second end of the metering valve stem projects from the metering valve body; and the resilient return member is arranged to apply a force to the second end of the metering valve stem.

In one or more embodiments, the resilient return member is an elastically deformable component having a rim, a hub, and at least one elastically deformable spoke; where the rim is fixed relative to the metering valve body and where the hub is axially moveable.

In one or more embodiments, the hub and the second end of the metering valve stem engage in a mating formation.

In one or more embodiments, the resilient return member is external to the metering chamber.

Alternatively the resilient return member is at least partially within the metering chamber.

In one or more embodiments, the resilient return member is unitary with the valve body.

According to a second aspect of the disclosure there is provided a metered dose inhaler valve, including:
a reservoir portion for assembly with a canister for receiving a pressurised formulation of medicament and propellant;
a metering valve body defining a metering chamber;
a metering valve stem having an outlet and being axially moveable within the metering valve body between a first position in which the metering chamber is in fluid communication with the pressurized canister, and a second position in which the metering chamber is in fluid communication with the valve stem outlet; and
where:
the metering valve body includes a first valve body part and a second valve body part, where the metering valve chamber is bounded by both the first valve body part and the second valve body part;
a first metering valve seal is defined by the first valve body part, the first metering valve seal including an opening through which the metering valve stem passes to form a dynamic seal between the metering valve stem and the outside atmosphere; and
the first metering valve seal opening is adapted to be stretched wider by the metering valve stem passing through it than it would be absent the valve stem.

Advantageously forming the chamber from two components allows the valve to be captured between them, avoiding the need for separate seal components to hold the valve stem in.

In one or more embodiments, the first valve body part and the second valve body part are arranged to capture the valve stem to constrain movement thereof between the first and second positions.

In one or more embodiments, the valve stem includes a collar configured to abut the first and second valve body parts to limit travel thereof.

In one or more embodiments, in the second position, the valve stem forms a second metering valve seal with the second valve body part, where the second metering valve seal opening is adapted to be stretched wider by the metering valve stem passing through it than it would be absent the valve stem.

In one or more embodiments, the valve stem includes a first section engaged with the first metering valve seal, a second section and a third section, where:
the second section is of a greater diameter than the third section;
in the first position, the third section of the valve stem is engaged in the second metering valve seal to allow fluid passage through the second metering valve seal; and
in the second position the second section of the valve stem is engaged in the second metering valve seal such that the second metering valve seal is stretched wider by the metering valve stem passing through it than it would be absent the valve stem.

In one or more embodiments, at least one of the first and second valve body parts extends into the other of the first and second valve body parts, thus forming an annular cavity therebetween, the annular cavity forming at least part of the metering chamber.

In one or more embodiments, the first and second body parts are engaged via an interference fit.

According to a third aspect of the present disclosure there is provided a metered dose inhaler valve, including:
a reservoir portion for assembly with a canister for receiving a pressurised formulation of medicament and propellant;
a metering valve body defining a metering chamber;
a metering valve stem having an outlet and being axially moveable within the metering valve body between a first position in which the metering chamber is in fluid communication with the pressurized canister, and a second position in which the metering chamber is in fluid communication with the valve stem outlet; and
where:
a first metering valve seal is defined by the metering valve body, the first metering valve seal including an opening formed by a radially inwardly projecting collar on a portion of the metering valve body, through which the metering valve stem passes to form a dynamic seal between the metering valve stem and the outside atmosphere; and
the first metering valve seal opening is adapted to be stretched wider by the metering valve stem passing through it than it would be absent the valve stem.

Advantageously, the provision of a collar provides a defined sealing surface which can be engineering to suit the application, and furthermore provides a degree of structural flexibility (in expansion) that allows for this type of “stretching” seal to take place.

In one or more embodiments, the radially inwardly projecting collar is formed on an axially extending projection of the metering valve body.

In one or more embodiments, the radially inwardly projecting collar is formed at a free end of the axially extending projection of the metering valve body.

In one or more embodiments, the axially extending projection of the metering valve body tapers to become smaller in cross-section towards the free end.

In one or more embodiments, the axially extending projection is frustoconical in shape.

In one or more embodiments, the collar is constructed from a plastic material having a Shore D hardness of at least 80.

The disclosure also provides a canister for receiving a pressurised formulation of medicament and propellant for use in a pressurized metered dose inhaler (pMDI), the canister including a metered dose inhaler valve according to any preceding aspect.

The disclosure provides a pressurised metered dose inhaler (pMDI) including:
a canister as defined above; and
an actuator including a stem socket for receiving the metering valve stem.

According to the disclosure there is provided a method of administering an aerosolised medicament from a pressurised metered dose inhaler (pMDI) including the steps of:
providing a pressurised metered dose inhaler as defined above; and
depressing the canister relative to the stem socket to move the metering valve stem from the first position to the second position to emit the contents of the metering valve chamber.

### BRIEF DESCRIPTION OF DRAWINGS

Embodiments of the present disclosure will now be described with reference to the following figures in which:

FIG. 1 is a side view of an inhaler including a canister containing a first valve according to the present disclosure;

FIG. 2 is a side view of a cross-section of the inhaler of FIG. 1 taken along the center line of the inhaler;

FIG. 3 is a detail perspective section view of the valve of the inhaler of FIG. 1;

FIG. 4 is a detail section view of the valve of the inhaler of FIG. 1;

FIG. 5 is an exploded view of the valve of the inhaler of FIG. 1;

FIGS. 6a & 6b are comparative detail section views of the valve of the inhaler of FIG. 1, where FIG. 6a illustrates the valve in a first position and FIG. 6b illustrates the valve in a second position; and

FIG. 7 is a detail section view of a part of a second valve according to the present disclosure.

### DESCRIPTION OF THE FIRST EMBODIMENT

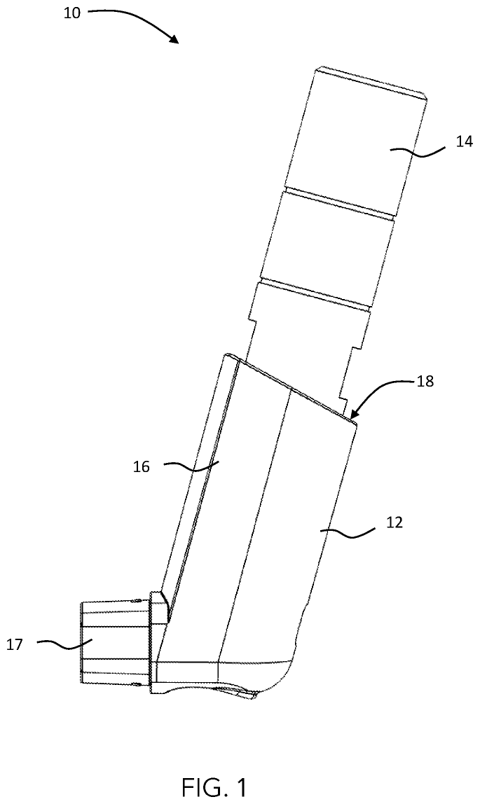

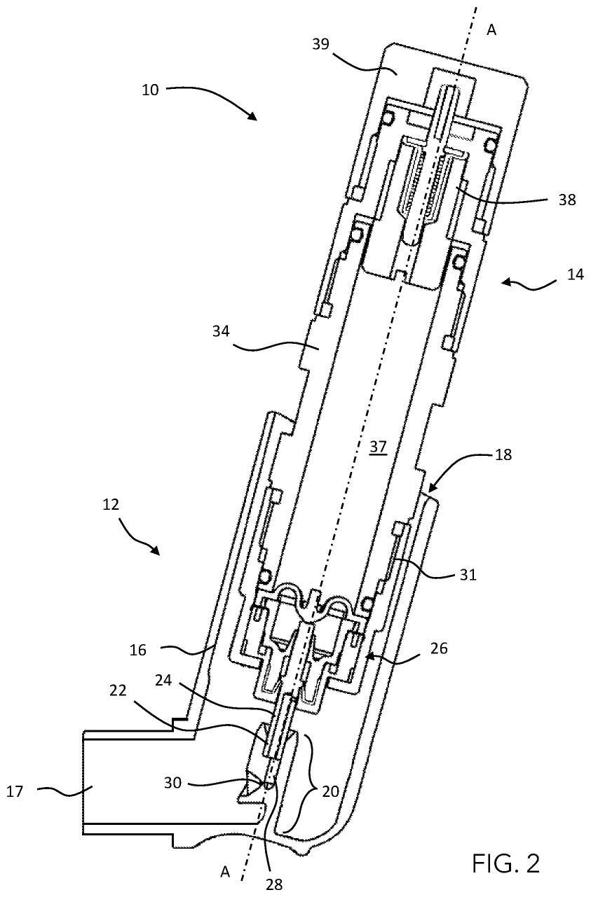

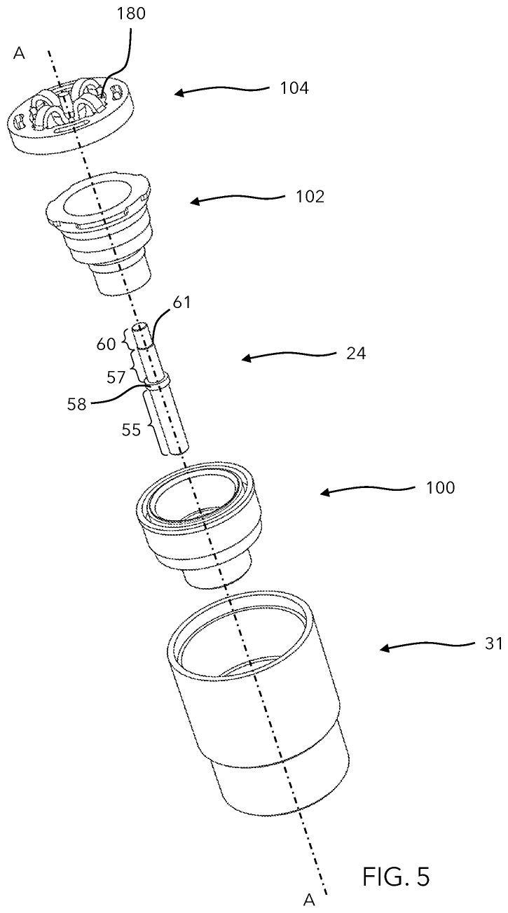

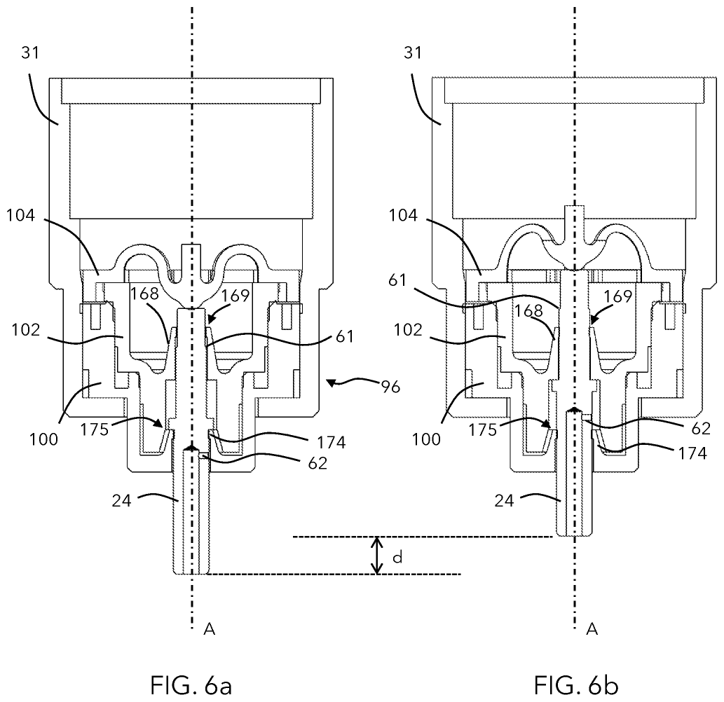

FIG. 1 depicts a pMDI 10 including an actuator 12 and a canister 14. In one or more embodiments, the canister 14 can be a pressurized canister. The pMDI 10 further includes a metered dose inhaler valve 26 (FIGS. 2-6) that includes a reservoir portion or cavity 37 for assembly with the canister 14 for receiving a pressurised formulation of medicament and propellant. In one or more embodiments, the valve 26 can be considered part of the canister 14. The valve 26 further includes a metering valve body 96 that defines a metering chamber 198, a metering valve stem 24 having an outlet 56 and being axially moveable within the metering valve body between a first position (FIG. 6a) in which the metering chamber is in fluid communication with the pressurized canister, and a second position (FIG. 6b) in which the metering chamber is in fluid communication with the valve stem outlet. The valve 26 can further include a resilient return member (e.g., spring) 104 urging the metering valve stem 24 from the second position to the first position. A metering valve seal is defined by the valve body 96 (e.g., defined by at least one of first collar 168 or second collar 174). The metering valve seal includes an opening (e.g., opening 169 of first collar 168 or opening 175 of second collar 174) through which the metering valve stem 24 passes to form a dynamic seal between the metering valve stem and the outside atmosphere. Further, the metering valve stem seal opening is adapted to be stretched wider by the metering valve stem 24 passing through it than it would be absent the valve stem.

The actuator 12 has a generally elongate actuator body 16 that acts as a housing for the canister 14. The canister 14 is inserted into canister opening 18 at the top of actuator 12. Canister 14 is pressurized and contains a medicament formulation for delivery to a user via actuator 12 and a mouthpiece 17 as will be described in further detail herein.

The medicament formulation includes one or more active pharmaceutical ingredients and a liquid propellant. The propellant acts to propel the formulation from the canister 14 into the mouthpiece 17 and then to the patient. The formulation can further include one or more excipients.

As is known in the art, the active pharmaceutical ingredient(s) and excipient(s) can be dissolved in the liquid propellant. Alternatively, the active pharmaceutical ingredient(s) and excipient(s) can be dispersed or suspended in the liquid propellant.

The medicament formulation can include any suitable pharmaceutical ingredients or medicinal compositions for the treatment of several conditions, in particular for the treatment of conditions of the respiratory system.

### Configuration

### Actuator

Turning now to FIG. 2, the actuator 12 includes body 16 having stem post 20. The stem post 20 has stem socket 22 for receiving metering valve stem 24 of metered dose inhaler valve (i.e., metering valve) 26. The stem post 20 also defines expansion chamber 28, which delivers medicament formulation into mouthpiece 17 via jet orifice 30.

### Canister—Housing

The canister 14 has a generally cylindrical form with a housing 34 sealed by a threaded connection 31 to form cavity or reservoir 37 for holding the propellant and medicament formulation. The housing 34 can include a pressure fill valve 38 to enable filling of the canister 14 with medicament formulation. The valve 38 is covered by a removeable cap 39. The cavity 37 and metering valve stem 24 share a central longitudinal axis A.

### Canister—Valve

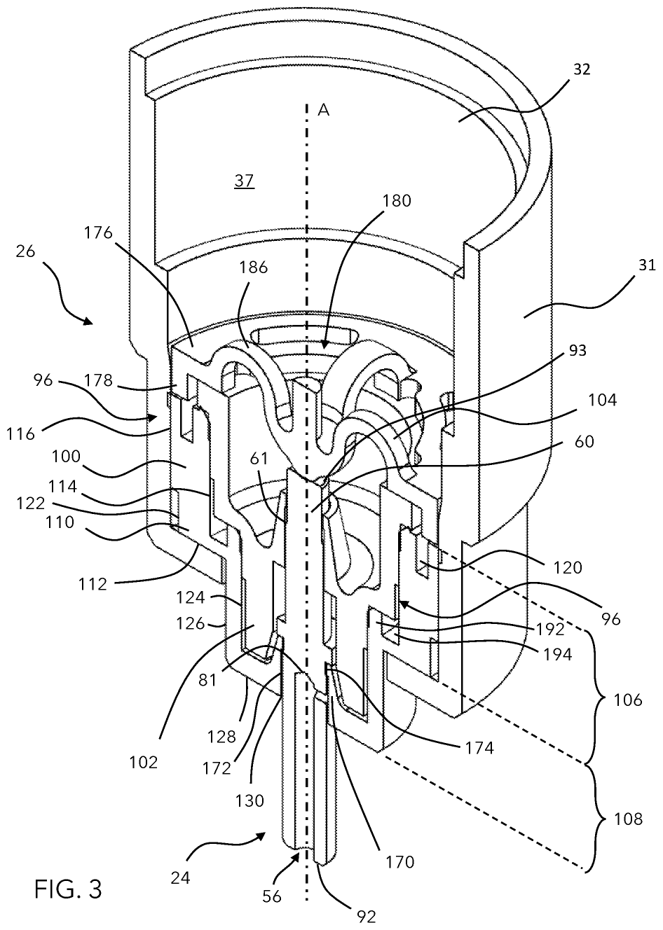

In FIGS. 3 to 5 the metering valve 26 is depicted in further detail.

The metering valve 26 includes a housing 34, the valve body 96 having a first valve body part 100, a second valve body part 102, the resilient return member (e.g., spring) 104 and the valve stem 24.

The first valve body part 100 and the second valve body part 102 are shown in cross section and can be axisymmetric, i.e., swept about a central axis (axis A).

### First Valve Body Part 100

Referring to FIG. 3, the first valve body part 100 is provided in the general shape of a stepped cylinder, having a first portion 106 and a second portion 108. The first portion 106 is of a greater inner and outer diameter than the second portion 108, thus providing a shoulder 110 having an annular surface 112 defined thereon.

The first portion 106 is generally cylindrical defining an inner surface 114 and an outer surface 116. An annular end surface 118 is provided at the end of the first portion 106 opposite to the shoulder 110. A circular groove 120 is provided in the end surface 118, extending in an axial direction. The outer surface 116 defines an inward step 122 at the end proximate the shoulder 110.

The second portion 108 is diametrically smaller than the first portion 106 and is also cylindrical. The second portion 108 defines an inner surface 124 and an outer surface 126. The second portion 108 also includes an endwall 128 having a circular port 130 defined in the centre thereof.

Projecting into the second portion 108 from the endwall 128 there is provided a hollow projection 170 forming a frustocone. The projection 170 defines a central through bore 172 contiguous with the port 130. At the free end of the projection 170 there is provided a radially inwardly projecting annular second collar 174. In one or more embodiments, the second collar 174 can define a metering valve seal.

Projecting axially into the first portion 106 from the inner diameter of the shoulder 110 there is provided a cylindrical abutment 192. The abutment 192 is inboard and spaced apart from the inner surface 114 of the first portion 106 such that a circular groove 194 is formed.

### Second Valve Body Part 102

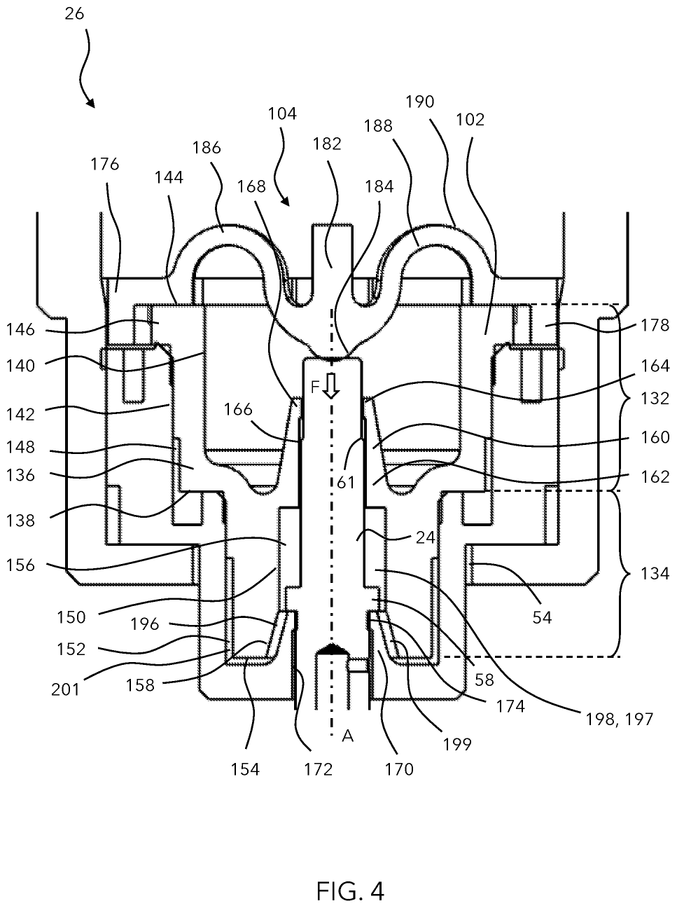

Referring to FIG. 4, the second valve body part 102 is also provided in the general shape of a stepped cylinder, having a first portion 132 and a second portion 134. The first portion 132 is of a greater inner and outer diameter than the second portion 134 thus providing a shoulder 136 having an annular surface 138 defined thereon.

The first portion 132 is generally cylindrical defining an inner surface 140 and an outer surface 142. An annular end surface 144 is provided at the end of the first portion 132 opposite to the shoulder 136. An annular radially outwardly extending tab 146 is provided proximate the end surface 118. The outer surface 142 defines an inward step 148 at the end proximate the shoulder 136.

The second portion 134 is diametrically smaller than the first portion 132 and is also cylindrical. The second portion 134 defines an inner surface 150 and an outer surface 152. The second portion 134 also includes annular end surface 154.

The second portion 134 defines an open bore 156 therethrough. The open bore 156 is generally cylindrical. At the free end of the second portion 134 the bore 156 tapers outwardly at a tapered region 158 to become wider at the end surface 154.

At the opposite end of the second portion 134, radially inward of the shoulder 136 there is provided a tapered, hollow projection 160 forming a frustocone directed into the first portion 132 (away from the second portion 134). The projection 160 has a first, wider end 162 joining the shoulder 136 and a narrower, free, second end 164 distal to the shoulder 136. The projection 160 defines a cylindrical through bore 166 contiguous with the bore 156. The projection 160 defines a radially inwardly projecting annular first collar 168 at the second end 164. In one or more embodiments, the first valve body part 100 and the second valve body part 102 are arranged to capture the valve stem 24 to constrain movement thereof between the first and second positions.

### Spring 104

Referring to FIGS. 3 and 4, the spring 104 includes an annular disc-like spring outer rim 176 defining an axially projecting circumferential foot 178. An opening 180 is provided in the centre of the spring 104. In the geometric centre of the opening 180 there is provided a central hub 182 which is generally cylindrical having a hemispherical tip 184. Connecting the hub 182 to the rim 176 there are provided a plurality of elastically deformable spokes 186 which are curved in cross-section. In this embodiment the spokes 186 have a concave surface 188 and a convex surface 190. There are 4 spokes, having openings therebetween. In one or more embodiments, the spring 104 is an elastically deformable component having the rim 176 and the hub 182, and at least one elastically deformable spoke 186, where the rim is fixed relative to the metering valve body, and where the hub is axially moveable. In one or more embodiments, the hub 182 and the second end 94 of the metering valve stem 24 engage in a mating formation. In one or more embodiments, the spring 104 is external to the metering chamber 198. Further, in one or more embodiments, the spring 104 is at least partially within the metering chamber 198.

The spring 104 can be constructed from a unitary plastics material.

### Stem 24

Referring to FIGS. 3 & 5, the valve stem 24 has a generally cylindrical shape having a first section 55, a second section 57, and a third section 60. The stem 24 further has an outlet 56 (FIG. 3) at its lower or first end 92 in fluid communication with a radial port 62 (FIGS. 6a-b) in the first section 55. The stem 24 has an outwardly extending circumferential abutment 58 where the first section 55 joins the second section 57. The first section 55 has a greater diameter than the second section 57. The second section 57 is of a greater diameter than the third section 60 and joined thereto by a bevelled section 61 at a second end 93. The second end 93 projects from the metering valve body 96.

The outlet 56 provides an outlet for the medicament formulation into the actuator 12. The radial port 62 extends from the outlet 56 though the wall of the metering valve stem 24 at a position proximate a base 81 of the outlet 56.

In terms of materials, the disclosure facilitates the use of high hardness (Shore D 80 and above measured by ASTM D2240-15 in a standard atmosphere of 23° C. and 50% relative humidity) materials to define the seal (e.g., materials utilized for at least one of the first collar 168 or second collar 174) between the valve body 96 and the stem 24. Therefore, in this embodiment, both the first and second valve body parts 100, 102 are constructed from acetal, also known as polyoxymethylene (POM), which advantageously can be machined, thereby easily facilitating the production (by turning) of such axisymmetric components. The stem 24 itself may be constructed from the same material, or a metal material such as steel. POM has a Shore D value of 80 to 95.

### Assembly

The valve stem 24 is assembled with the second valve body part 102 by passing the third section 60 through the second portion 134 and through the bore 166 to project from the projection 160.

The first valve body part 100 is now assembled with the second valve body part 102, such that the second portion 134 of the latter engages with the second portion 108 of the former. Similarly, the first portion 132 of the second valve body part 102 engages the first portion 106 of the first valve body part 100. Travel of the second valve body part 102 into the first valve body part 100 is limited by the abutment 192 which bears against the annular surface 138 of the shoulder 136. The first valve body part 100 and second valve body part 102 are dimensioned such that an interference fit is formed therebetween, meaning once assembled they are effectively inseparable. The interference fit exists between the respective first portions 106, 132 and the respective second portions 108, 134.

When engaged as described herein, the metering chamber 198 is formed between, and partially bounded by, each of the first and second valve body parts 100, 102. With reference to FIG. 4, the metering chamber 198 has several portions, including a cylindrical portion 197, which at its lower end is in fluid communication with a conical channel 196 formed between the inner surface of the tapered region 158 of the bore 156 and the outer surface of the projection 170 (which is partially engaged therewith). The conical channel 196 is in fluid communication with an annular portion 199, which in turn is in communication with an outer cylindrical portion 201. The metering chamber 198 is therefore substantially U-shaped.

The valve stem 24 is thus engaged with both the first valve body part 100 and the second valve body part 102 and able to move along the axis A between the position shown in FIG. 6a (first or rest position) and the position shown in FIG. 6b (second or actuated position). In one or more embodiments, at least one of the first collar 168 or second collar 174 can define a metering valve seal that includes an opening through which the metering valve stem 24 passes to form a dynamic seal between the metering valve stem and the outside atmosphere. In one or more embodiments, the first valve body part 100 can define a first metering valve seal that includes an opening 175 (FIG. 6a) through which the metering valve stem passes to form a dynamic seal between the metering valve stem 24 and the outside atmosphere. In one or more embodiments, the valve stem 24, when in the second position, can form a second metering valve seal with the second valve body part 102. An opening 169 (FIG. 6a) formed by the first collar 168 is configured such that it allows passage of the third section 60 of the stem 24 with a clearance gap therebetween but is completely filled (sealed) by the second section 57. The opening 169 formed by the first collar 168 is adapted to be stretched wider by the second section 57 of the valve stem 24 passing through it than it would be absent the valve stem. The opening 175 formed by the second collar 174 is adapted to be stretched wider by the first section 55 of the valve stem 24 passing through it than it would be absent the valve stem. In one or more embodiments, the first section 55 of the valve stem 24 is engaged with the first metering valve seal formed by the first valve body part 100 (e.g., second collar 174). When the valve stem 24 is in the first position, the third section 61 of the valve stem is engaged in the second metering valve seal formed by the second valve body part 102 (e.g., first collar 168) to allow fluid passage through the second metering valve seal (FIG. 6a). Further, when the valve stem 24 is in the second position, the second section 57 of the valve stem 24 is engaged in the second metering valve seal defined by the second valve body part 102 such that the second metering valve seal is stretched wider by the metering valve stem passing through it than it would be absent the valve stem (FIG. 6b).

The spring 104 is assembled with the valve body parts 100, 102 and the stem 24 by fixing the rim 176 such that it is in contact with the surface 144 of the second valve body part 102. The axially projecting circumferential foot 178 engages the surface 118 of the first valve body part 100. The hemispherical tip 184 of the hub 182 extends into contact with the second end 94 of the stem 24. At rest (FIG. 4) the spring 104 is either in an undeformed or lightly deformed state providing a force F along axis A onto the stem 24. In one or more embodiments, the spring 104 is arranged to apply the force F to the second end 94 of the metering valve stem 24. The stem 24 cannot move any further from the valve 26 due to the contact of the abutment 58 against the top of the projection 170.

The valve 26 as described above is assembled with the ferrule 32 such that the second portion 134 engages with, and projects from, the port 54. The housing 34 and ferrule 32 are assembled as shown in FIG. 2 and the cavity 37 filled with a propellant, medicament(s) and optionally excipient(s) in a fluid (pressurised liquid) form. In this embodiment, the housing 34 and ferrule 32 are assembled with a threaded connection 31.

It will be noted that the cavity 37 is in fluid communication with the valve 26. The contents of the cavity 37 are able to flow between the top of the stem 24 and the projection 160 into the bore 156. As well as filling the bore 156, the contents are also able to fill the conical channel 196, and the voids between the respective second portions 108, 134 of the second valve body part 102 and first valve body part 100. These areas (downstream of the projection 160) form the metering chamber 198.

The canister 14 is then assembled with the actuator 12 with the first end 92 of the valve stem 24 engaged with the stem post 20.

### Use

In use, the user depresses the base of the canister 14 which in turn forces the valve stem 24 into the canister 14. This moves the stem 24 from the rest position (i.e., first position) of FIG. 6a to the actuated position (i.e., second position) of FIG. 6b. When the valve stem 24 is in the first position, the metering chamber 198 is in fluid communication with the pressurized canister 14. And when the valve stem 24 is in the second position, the metering chamber 198 is in fluid communication with the valve stem outlet 56. Comparing FIGS. 6a and 6b, the stem 24 has been pushed into the valve body 96 by an actuation distance d. This has the effect of both:
a) preventing further flow from the cavity 37 into the metering chamber 198 as the second section 57 of the valve stem 24 below the bevelled section 61 seals against the first collar 168. The second section 57 of the valve stem 24 and the first collar 168 form a cylindrical contact surface parallel to the main axis A, and furthermore are in sliding contact in the sealed condition. The bevelled section 61 moves completely past, and clears, the first collar 168; and
b) raising the port 62 (and therefore outlet 56) above the second collar 174 into fluid communication with the metering chamber 198, allowing the pressurised contents to flow from the valve stem outlet 56 of the valve stem 24 into the actuator 12.

This depression of the valve stem 24 by the user acts to elastically deform the spring 104, resulting in the restoring force F increasing. When the user releases the canister 14, the restoring force is sufficient to move the canister to its first position relative to the stem (i.e., to push the stem 24 from the canister). This action moves the port 62 below the second collar 174 therefore isolating the metering chamber 198 from the outside and opening the channel from the cavity 37 to the metering chamber 198.

### Experiments

Two valves were fabricated, in Valve 1 all components (valve inner, valve outer, valve stem and spring) were constructed from polyoxymethylene. For Valve 2 all components were also made using polyoxymethylene except for the stem which was made from stainless steel. The valves were connected to canisters. The canisters were filled with approximately 9 grams of HFA 134a and the leak rate of the valves was determined gravimetrically over a period of 43 hours. The leak rate for Valve 1 (polyoxymethylene stem) was 428 mg/year and for Valve 2 (stainless steel stem) was 265 mg/year.

### DESCRIPTION OF THE SECOND EMBODIMENT

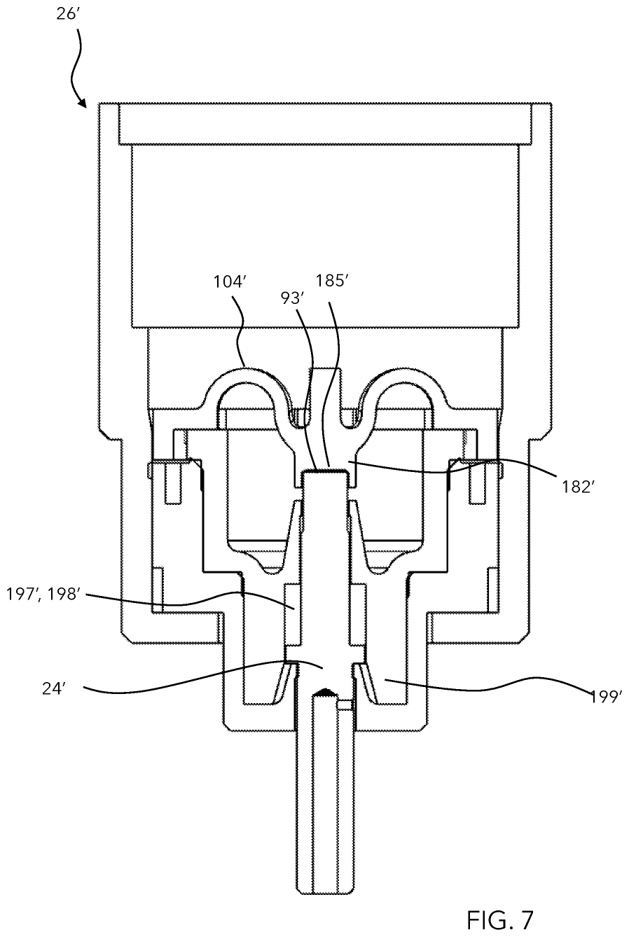

Referring to FIG. 7, a second embodiment of a metering valve 26′ is shown. The main difference between first and second embodiments is the spring 104′ of the second embodiment, which instead of defining a hemispherical tip 184, defines a recess 185′ being a closed cylindrical bore for receiving the end of the valve stem 24′ such that a hub 182′ of the spring and a second end 93′ of the valve stem engage in a mating formation. This reduces the effect of side loading on the stem 24′, ensuring engagement with the spring.

A further difference is that the metering chamber 198′ includes the cylindrical portion 197′ and the conical portion 199′, but not the annular or outer cylindrical portions of the first embodiment. This is beneficial in certain circumstances, as it can reduce the likelihood of medicament build-up in those areas.

### Variations

In other embodiments the mouthpiece 17 can be replaced by a nosepiece (not depicted) to enable nasal delivery.

Although one type of spring is described herein, other resilient structures and elements are possible, including but not limited to coil springs, compression springs, tension springs, leaf springs, torsion springs etc. The spring may be located within the metering chamber (rather than outside it).

In the above embodiments, both the seal between the valve stem and the canister cavity (the upper seal) and the seal between the valve stem and the outside of the canister (the lower seal) are formed with components having a high hardness. It will be noted that such a seal may be provided at only one of these locations, and the other provided with a seal having e.g., a lower hardness material.

The ferrule and the valve outer could be moulded as one component (resulting in a 4-component assembly).

The ferrule design can be altered to permit attachment to a canister via crimping or via a screw thread to the canister body.

In an alternative embodiment, clipping/push-fit features are included in the spring design to allowing fixing to the valve inner body.

The spring design could be altered to allow it to become a feature of the first valve body part 100 to produce a 4-component assembly. If the spring and valve inner were combined, and the ferrule and valve outer were combined, this would provide a 3-component assembly.

All references and publications cited herein are expressly incorporated herein by reference in their entirety into this disclosure, except to the extent they may directly contradict this disclosure. Illustrative embodiments of this disclosure are discussed, and reference has been made to possible variations within the scope of this disclosure. These and other variations and modifications in the disclosure will be apparent to those skilled in the art without departing from the scope of the disclosure, and it should be understood that this disclosure is not limited to the illustrative embodiments set forth herein. Accordingly, the disclosure is to be limited only by the claims provided below.

1. A metered dose inhaler valve, comprising:
a reservoir portion for assembly with a canister for receiving a pressurised formulation of medicament and propellant;
a metering valve body defining a metering chamber;
a metering valve stem having an outlet and being axially moveable within the metering valve body between a first position in which the metering chamber is in fluid communication with the pressurized canister, and a second position in which the metering chamber is in fluid communication with the valve stem outlet; and
a resilient return member urging the metering valve stem from the second position to the first position;
wherein:
a metering valve seal is defined by the valve body, the metering valve seal comprising an opening through which the metering valve stem passes to form a dynamic seal between the metering valve stem and the outside atmosphere; and
the metering valve stem seal opening is adapted to be stretched wider by the metering valve stem passing through it than it would be absent the metering valve stem.

2. A metered dose inhaler valve according to claim 1, wherein:
the metering valve stem has a first end and a second end;
the outlet is defined at the first end;
the second end of the metering valve stem projects from the metering valve body; and
the resilient return member is arranged to apply a force to the second end of the metering valve stem.

3. A metered dose inhaler valve according to claim 2, wherein the resilient return member is an elastically deformable component having a rim, a hub, and at least one elastically deformable spoke; wherein the rim is fixed relative to the metering valve body and wherein the hub is axially moveable.

4. A metered dose inhaler valve according to claim 3, wherein the hub and the second end of the metering valve stem engage in a mating formation.

5. A metered dose inhaler valve according to claim 1, wherein the resilient return member is external to the metering chamber.

6. A metered dose inhaler valve according to claim 1, wherein the resilient return member is at least partially within the metering chamber.

7. (canceled)

8. A metered dose inhaler valve, comprising:
a reservoir portion for assembly with a canister for receiving a pressurised formulation of medicament and propellant;
a metering valve body defining a metering chamber;
a metering valve stem having an outlet and being axially moveable within the metering valve body between a first position in which the metering chamber is in fluid communication with the pressurized canister, and a second position in which the metering chamber is in fluid communication with the valve stem outlet; and
wherein:
the metering valve body comprises a first valve body part and a second valve body part, wherein the metering valve chamber is bounded by both the first valve body part and the second valve body part;
a first metering valve seal is defined by the first valve body part, the first metering valve seal comprising an opening through which the metering valve stem passes to form a dynamic seal between the metering valve stem and the outside atmosphere; and
the first metering valve seal opening is adapted to be stretched wider by the metering valve stem passing through it than it would be absent the valve stem.

9. A metered dose inhaler valve according to claim 8, wherein the first valve body part and the second valve body part are arranged to capture the valve stem to constrain movement thereof between the first and second positions.

10. A metered dose inhaler valve according to claim 9, wherein the valve stem comprises a collar configured to abut the first and second valve body parts to limit travel thereof.

11. A metered dose inhaler valve according to claim 9, wherein in the second position, the valve stem forms a second metering valve seal with the second valve body part, wherein the second metering valve seal opening is adapted to be stretched wider by the metering valve stem passing through it than it would be absent the valve stem.

12. A metered dose inhaler valve according to claim 11, wherein the valve stem comprises a first section engaged with the first metering valve seal, a second section and a third section, wherein:
the second section is of a greater diameter than the third section;
in the first position, the third section of the valve stem is engaged in the second metering valve seal to allow fluid passage through the second metering valve seal; and
in the second position the second section of the valve stem is engaged in the second metering valve seal such that the second metering valve seal is stretched wider by the metering valve stem passing through it than it would be absent the valve stem.

13. A metered dose inhaler valve according to claim 8, wherein at least one of the first and second valve body parts extends into the other of the first and second valve body parts, thus forming an annular cavity therebetween, the annular cavity forming at least part of the metering chamber.

14. (canceled)

15. A metered dose inhaler valve, comprising:
a reservoir portion for assembly with a canister for receiving a pressurised formulation of medicament and propellant;
a metering valve body defining a metering chamber;
a metering valve stem having an outlet and being axially moveable within the metering valve body between a first position in which the metering chamber is in fluid communication with the pressurized canister, and a second position in which the metering chamber is in fluid communication with the valve stem outlet; and
wherein:
a first metering valve seal is defined by the metering valve body, the first metering valve seal comprising an opening formed by a radially inwardly projecting collar on a portion of the metering valve body, through which the metering valve stem passes to form a dynamic seal between the metering valve stem and the outside atmosphere; and
the first metering valve seal opening is adapted to be stretched wider by the metering valve stem passing through it than it would be absent the valve stem.

16. A metered dose inhaler valve according to claim 15, wherein the radially inwardly projecting collar is formed on an axially extending protrusion of the metering valve body.

17. A metered dose inhaler valve according to claim 16, wherein the radially inwardly projecting collar is formed at a free end of the axially extending projection of the metering valve body.

18. A metered dose inhaler valve according to claim 17, wherein the axially extending projection of the metering valve body tapers to become smaller in cross-section towards the free end.

19. A metered dose inhaler valve according to claim 18, wherein the axially extending projection is frustoconical in shape.

20. A metered dose inhaler valve according to claim 8, wherein the collar is constructed from a plastic material having a Shore D hardness of at least 80.

21. A canister for receiving a pressurised formulation of medicament and propellant for use in a pressurized metered dose inhaler (pMDI), the canister comprising a metered dose inhaler valve according to claim 1.

22. A pressurised metered dose inhaler (pMDI) comprising:
a canister according to claim 21; and
an actuator comprising a stem socket for receiving the metering valve stem.
